(19) United States
(12) Patent Application Publication
(10) Pub. No.: US 2007/0275943 A1
(43) Pub. Date: Nov. 29, 2007

(54) Method and Composition for Treatment or Prophylaxis of Amyloidosis Disorders

(75) Inventors: Timothy Matthias Morgan, Victoria (AU); Nina Frances Wilkins, Victoria (AU)

Correspondence Address: FOLEY AND LARDNER LLP;SUITE 500, 3000 K STREET NW, WASHINGTON, DC, 20007, US

(73) Assignee: Acrux DDS Pty Ltd.

(21) Appl. No.: 10/579,754

(22) PCT Filed: Nov. 19, 2004
(86) PCT No.: PCT/AU04/01610

§ 371 Date: May 8, 2007

Related U.S. Application Data
(60) Provisional application No. 60/523,139, filed on Nov. 19, 2003.

(51) Int. Cl. A61K 31/47; A61K 31/192; A61K 31/56; A61K 31/60; A61K 9/12; A61P 25/28

(52) U.S. Cl. 514/182; 424/45; 514/292; 514/312; 514/576

(57) ABSTRACT

A method of treatment or prophylaxis of amyloidosis disorders in a patient the method comprising topically applying to an area of skin of the patient a composition comprising: one or more zinc chelators; and one or more dermal penetration enhancers.

### FIELD

The present invention relates to compositions for the administration of zinc chelators such as 1,10-phenanthroline and to use of such compositions for the prevention and treatment of amyloidosis disorders.

### BACKGROUND

Amyloidosis is not one disease but a diverse group of diseases of acquired or hereditary origin and characterized by the extracellular deposition of one of several different types of protein fibrils with similar properties and called amyloid. Amyloid deposition may be either a primary (idiopathic) process without known antecedent or secondary to some other condition and may be localized to one specific site or generalized throughout the body (systemic). Amyloid deposits cause a number of common and rare diseases and there are many different amyloid proteins that can be involved. For example, Alzheimer's disease and Creutzfeldt-Jakob disease are two distinct conditions characterized by amyloid deposits in the brain, but the proteins involved are different.

A major component of the amyloid deposits in Alzheimer's disease is a polypeptide referred to herein as Aβ (Amyloid-beta). Aβ also accumulates in the wall and the lumen of the brain vessels. The major form of Alzheimer's disease is sporadic and has a late onset, whereas a small percentage of cases are familial and have an early onset. Some of the familial cases of Alzheimer's disease are strongly associated with one or more mutations at different sites on the Aβ precursor protein, the gene of which lies on chromosome 21. Whether these mutations are the cause of Alzheimer's disease in the affected patients, however, has not been proven experimentally.

The plaques are not unique to Alzheimer's disease. The senile plaques are also seen in Down syndrome and in both aged human and animal brains. The numbers of plaques in non-demented aged humans are sometimes similar to those seen in Alzheimer's disease cases (Katzman et al., 1988, Ann. Neurol. 23:138-144).

The precipitation of synthetic Aβ has been shown to be caused by several environmental factors including low pH, high salt concentrations and the presence of metals, e.g., zinc, copper, and mercury (Bush et al., 1995, Science 268:1921-1923). It has been reported that Aβ itself specifically and saturably binds zinc with a high affinity binding (K_{D}=107 nM) at a molar ratio of 1:1 (zinc: Aβ) (Bush et al., 1994, J. Biol. Chem. 269:12152-12158). This binding takes place at physiological concentrations of zinc (Bush et al., 1994, Science 265:1464-1467).

There is a strong supposition that the removal of amyloid deposits from patients suffering from Alzheimer's disease will alleviate the symptoms of Alzheimer's disease. Therefore, several attempts have been made to prepare a drug for the removal of amyloid deposits, as methods for healing Alzheimer's disease are urgently sought.

International Publication No. WO 93/10459, dated May 27, 1993, discloses a method for the treatment of Alzheimer's disease by administering a zinc binding agent. As preferred compounds, phytic acid, desferri-oximine, sodium citrate, EDTA, 1,2-diethyl-3-hydroxy-pyridin-4-one, and 1-hydroxyethyl-3-hydroxy-2-methyl-pyridin-4-one are mentioned.

German publication DE 39 32 338, dated Apr. 11, 1991, discloses the use of an aluminum chelator, such as 8-hydroxy-quinoline, for the treatment of Alzheimer's disease.

U.S. Pat. No. 5,373,021, dated Dec. 13, 1994, discloses disulfiram and its salts and analogs. According to this patent, disclosed compounds may be used to reduce neurological damage caused by Alzheimer's disease.

The hitherto known compounds suggested for the treatment of Alzheimer's disease have several drawbacks, which has prevented their widespread use. Many of the compounds are unable to penetrate the blood-brain-barrier and thus cannot readily reach the areas in which the amyloid is deposited. Disulfiram, which may penetrate the blood-brain-barrier, has the drawback that when it is combined by a patient with ethyl alcohol, it causes severe adverse reactions, including headaches, nausea, vomiting, sweating, thirst, weakness, and low blood pressure.

A number of zinc chelators have been found such as clioquinol which cross the blood-brain barrier. However potentially useful drugs have severe side effects. The Japanese Government officially banned the sale of clioquinol in September 1970. The ban was motivated by the presumption that clioquinol caused subacute myelo-optico-neuropathy (SMON). Subsequently, clioquinol (at that time used as a treatment for gastrointestinal dysfunction) was withdrawn from the market in most other countries of the world on the recommendation of the World Health Organization.

SMON develops with an acute or subacute onset preceded by abdominal disorders and is characterized by dysesthesia of the legs, sensory disturbances, a variable degree of motor weakness, and visual loss. Corresponding to these clinical findings, SMON reveals pathologically symmetrical degeneration in peripheral nerves, spinal cord, posterior column, cardiac-spinal tract, and optic nerves.

The occurrence of SMON was confined to Japan even though clioquinol was prescribed worldwide and not only in Japan. In the published literature no systematic pathological features resulting from the administration of clioquinol have been described other than the cases of SMON in Japan.

U.S. Pat. No. 5,487,884 describes the use of certain chelating agents to reduce skin-ageing effects of exposure to ultraviolet radiation The chelators referred to includeo 2,2′-dipyridylamine; 1,10-phenanthroline; di-2-pyridylketone; 2-furildioxime; 2,3-bis(2-pyridyl)pyrazine; 1-hydroxy4-methyl-6-(2,4,4-trimethylpentyl)-2(1H)-pypyridone; 2,3-dihydroxybenzoic acid; ethylenediamine-N,N-bis(2-hydroxyphenylacetic acid), dimethyl ester; 1,1′-carbonyldiimidazole; 1,2-dimethyl-3-hydroxypyrid-4-one; 2,4,6-tri(2-pyridyl)-1,3,5-triazine; 1-pyrrolidinecarbodithioic acid; diethyldithiocarbamic acid; 6-cyclohexyl-1-hydroxy-4-methyl-2(1H)pyridinone; 2,2′-dipyridyl; 1,2-cyclohexanedione dioxime; 3-hydroxy-2-methyl-4-pyrone; 2,3-bis(2-pyridyl)-5,6-dihydropyrazine; 3-(4-phenyl-2-pyridyl)-5-phenyl-1,2,4-triazine; 5-hydroxy-2-(hydroxymethyl)-4H-pyran-4-one; 2,3-dihydroxypyridine; 2,2′-biquinoline; 2,2′-bipyrazine; 3-(2-pyridyl)5,6-diphenyl-1,2,4-triazine; 4,4′-dimethyl-2,2′-dipyridyl; 4,5-dihydroxy-1,3-benzene-disulfonic acid; phenyl 2-pyridyl ketoxime; desferrioxamine B; 5,7-dichloro-8-hydroxyquinoline; 2,3-dihydroxynaphthalene; 2,3,5,6-tetrakis-(2′-pyridyl)pyrazine; 2,4-bis(5,6-diphenyl-1,2,4-triazine-3-yl)pyridine; di-2-pyridyl glyoxal; 6-hydroxy-2-phenyl-3(2H)-pyridazinone; 2,4-pteridinediol; 3-(4-phenyl-2-pyridyl)-5,6-diphenyl-1,2,4-triazine; N-benzoyl-N-phenylhydroxylamine; 3-amino-5,6-dimethyl-1,2,4-triazine; 2,6-pyridinedicarboxylic acid; 2,4,5-trihydroxypyrimidine; and 4-(2-amino-1-hydroxyethyl)-1,2-benzenediol.

U.S. Pat. No. 6,001,852 studies the effect of zinc chelators and reports that the significant class—(non-specific metal chelation) and drug specific—(SMON, subacute myelo-optico-neuropathy) side effects which need to be inhibited by using a combination of intermittent therapy to provide a “wash out period” of one to four weeks to reduce unwanted side effects and combination therapy with vitamin B12 therapy.

There is a need for a zinc chelator composition and method of treatment using a zinc chelator which will allow effective delivery across the blood-brain barrier with more effective control of side effects.

No admission is made that any reference, including any patent or patent document, cited in this specification constitutes prior art. In particular, it will be understood that, unless otherwise stated, reference to any document herein does not constitute an admission that any of these documents forms part of the common general knowledge in the art in Australia or in any other country. The discussion of the references states what their authors assert, and the applicant reserves the right to challenge the accuracy and pertinency of any of the documents cited herein.

### SUMMARY

We have found that zinc chelators such as 1,10-phenanthroline can be administered transdermally to provide effective control of level of zinc in the circulation. The choice of dermal penetration enhancer and the zinc chelators enable the dose of zinc chelator to be sustained at a low level to significantly reduce or avoid any clinically significant non-specific chelation of other metals within the body.

Accordingly, in a first aspect the present invention provides a method of treatment or prophylaxis of amyloidosis disorders in a patient the method comprising topically applying to an area of skin of the patient a composition comprising:

one or more zinc chelators; and

one or more dermal penetration enhancers.

Preferably the composition will also contain a volatile pharmaceutically acceptable solvent.

In a second aspect the invention provides the use of a zinc chelator in preparation of a transdermal composition for treatment of amyloidosis disorders by topical application to the skin of a patient.

In a third aspect the invention provides a composition for treatment or prophylaxis of amyloidosis disorders the composition comprising:

one or more zinc chelators;

one or more dermal penetration enhancers; and

preferably also a volatile pharmaceutically acceptable solvent.

The composition of the invention may and preferably will contain one or more estrogens, such as estradiol. The presence of one or more estrogens in combination with zinc chelators provides a further benefit in the prevention and treatment of amyloidosis disorders such as Alzheimer's disease.

### DETAILED DESCRIPTION

The present invention uses one or more dermal penetration enhancers for enhanced transdermal drug delivery. The invention may use traditional dosage forms such as gels, lotions and patches.

Preferably the composition is applied by spraying the composition onto the skin of the patient. In addition to providing improved percutaneous absorption efficiency, the topical spray application of the composition of the invention in many cases provides lower irritancy than more occlusive delivery methods such as transdermal patches, because the composition is non-occlusive to the skin.

In drug delivery compositions according to the present invention one or more other components selected from the group consisting of active agents, co-solvents, surfactants, emulsifiers, antioxidants, preservatives, stabilisers, diluents and mixtures of two or more of said components may be incorporated as is appropriate to the particular route of administration and dosage form. The amount and type of components used should be compatible with the dermal penetration enhancers of this invention as well as with the zinc chelating agent. A co-solvent or other standard adjuvant, such as a surfactant, may be required to maintain the zinc chelating agent in solution or suspension at the desired concentration.

In each of the above cases the amount of zinc chelator used may be minimised to avoid non-specific chelation of other physiologically relevant metals within the body, notwithstanding that it is envisaged that the present invention could also contain even more specific zinc chelators. One of the significant advantages of the present invention is that it provides a sustained relatively low dose of zinc chelator which may be used to reduce Aβ deposits while avoiding or reducing the incidence of the serious side effects previously reported. As the transdermal administration of this class of drug had not been reported it was not expected that the combination of features required for effective transdermal administration, transport across the blood brain barrier and solubilisation of Aβ deposits could be met.

### BRIEF DESCRIPTION OF THE FIGURES

In the accompanying figures:

FIG. 1 Shows the cumulative amount of 1,10-phenanthroline penetrating across human epidermis (μg/cm²) versus time (hours) for a transdermal spray composition with or without the dermal penetration enhancer, octyl salicylate (octisalate). Error bars represent SEM.

FIG. 2 Shows the cumulative amount of estradiol penetrating across human epidermis (μg/cm²) versus time (hours) for a transdermal spray composition with or without the dermal penetration enhancer, octyl salicylate (octisalate). Error bars represent SEM.

In describing the present invention, the following terminology will be used in accordance with the definitions set out below.

The terms “topical” and “transdermal” are used herein in the broadest sense to refer to administration of a drug to the skin surface or mucosal membrane of an animal, including humans, so that the drug passes through the skin tissue and/or into the animal's blood stream, thereby providing a local or systemic effect. The term transdermal is intended to include transmucosal drug administration i.e. administration of a drug to the mucosal surface of an animal so that the drug passes through the mucosal tissue and into the blood stream. Unless otherwise stated or implied, the terms topical drug delivery and transdermal drug delivery are used interchangeably. Where used herein the terms transdermal and dermal administration include transmucosal and mucosal administration. These phrases will of course also embrace administration via other types of dermis such as the skin.

The term “stratum comeum” is used herein in its broadest sense to refer to the outer layer of the skin, which is comprised of (approximately 15) layers of terminally differentiated keratinocytes made primarily of the proteinaceous material keratin arranged in a ‘brick and mortar’ fashion with the mortar being comprised of a lipid matrix made primarily from cholesterol, ceramides and long chain fatty acids. The stratum corneum creates the rate-limiting barrier for diffusion of the active agent across the skin.

The term “dermal penetration enhancer” is used herein in its broadest sense to refer to an agent which improves the rate of percutaneous transport of active agents across the skin or mucosa or use and delivery of active agents to organisms such as animals, whether it be for local application or systemic delivery.

The term “non-occlusive” is used herein in its broadest sense to refer to not trapping or closing the skin to the atmosphere by means of a patch device, fixed reservoir, application chamber, tape, bandage, sticking plaster, or the like which remains on the skin at the site of application for a prolonged length of time.

The composition of the present invention preferably contains from about 0.1% to about 10% of a zinc chelator, from about 0.1% to about 10% of a dermal penetration enhancer, and from about 45% to about 99.8% of a volatile solvent, and optionally from about 0.1% to about 2% of an estrogen.

In another preferred form the volatile liquid is ethanol, isopropanol or mixture thereof in the range of about 80 to 98%. More preferably the composition of the invention will comprise 1 to 5% of a zinc chelator, from about 2 to 8% of the dermal penetration enhancer, from about 45 to 90% ethanol, isopropanol or mixture thereof, 5 to 45% water; and optionally 0.5 to 5% of a thickening agent.

Suitable zinc chelators are those having a structure amenable to transdermal drug delivery and with sufficient lipid and water solubility to remove zinc from amyloid deposits to allow the re-solubilization of Aβ deposits and/or the prevention of their formation. Suitable structures of zinc chelators being those that have preferably a molecular weight less than 500 Daltons, a melting point less than 200 degrees Celcius, less than or equal to 3 hydrogen bond donors, an octanol-water partition coefficient between 1 and 4 and a water solubility greater than 10 microgram per millilitre. Preferred chemical classes of such suitable zinc chelators are phenanthrolines and their derivatives, such as 1,10 phenanthroline, aryl propionic acids and their derivatives, such as ibuprofen and flurbiprofen, and any other compounds fitting the previously defined physicochemical properties (molecular weight less than 500 Daltons, a melting point less than 200 degrees Celcius, less than or equal to 3 hydrogen bond donors, an octanol-water partition coefficient between 1 and 4 and a water solubility greater than 10 microgram per millilitre) and shown to have a chemical binding site or sites(s) for a zinc ion as determined by a negative binding energy of greater than 20 kcal/mole for the association of the zinc ion and the compound of interest when using a recognised 3-dimensional molecular modelling software such as “ChemDraw” 3D, version 5.0 running a MM2 force-field for the steric energy calculation.

Suitable zinc chelating agents include, but are not limited to, 3-mercapto-D valine, bis(diethylthiocarbamoyl) disulfide, N,N,N′,N′-tetrakis (2-pyridylmethyl)-ethylenediamine,N-(6-methoxy-8-quinolyl)-p-toluenesulfonamide, 8-hydroxy quinoline, 8-hyroxy quinoline-5-sulphonic acid, diethyl dithiocarbamate, phenanthroline and it's derivatives, dipicolinate, diphenylthiocarbazone, dithizone, cimetidine, dipicolinic acid, clioquinol or pharmaceutically acceptable salts or derivatives of any one of the aforementioned.

Additional zinc chelating agents include, but are not limited to diclofenac, ibuprofen, naproxen, piroxicam, indomethacin, ketoprofen, nabumetone, apazone, sulindac, meloxicam, tiaprofenic acid, flurbiprofen, tolfenamic acid, phenylbutazone, benzydamide, aspirin, salicylic acid or pharmaceutically acceptable salts or derivatives of any one of the aforementioned. While a number of these drugs are known for treatment of other pharmaceutical indications the dose required in treatment of amyloidosis disorders is typically different (often lower) than their more common use.

The preferred zinc chelator for use in the composition and method of the invention is 1,10-phenanthroline.

The concentration of zinc chelator and the dose of composition applied will be sufficient to provide an effective blood concentration of zinc chelator having regard to the specific formulation and the area of topical administration.

The dose of zinc chelator required to provide optimal treatment of amyloidosis or protection against the development of amyloidosis disorders will depend upon the nature of the chelator and its properties. The relevant properties include the effectiveness of chelation of metals such as zinc and the efficiency with which the chelator crosses the blood brain barrier. In addition, the performance of the dermal penetration enhancer to deliver a desired chelating agent varies with differences in both the nature of the dermal penetration enhancer and the chelator. It is understood that different dermal penetration enhancers may need to be selected to be appropriate for delivery of various metal chelators. Preferably, the release rate profile of the chelating agent into the systemic circulation is approaching zero order in nature so as to reduce potential side effects associated with elevated maximum concentration (Cₘₐₓ) to average concentration (C_{avg}) ratios often seen with alternative dosage forms. Preferably the composition of the invention is applied to provide a therapeutically effective blood serum level over 12 hours and more preferably over 24 hours.

The dermal penetration enhancer may be selected from the classes of enhancers that are lipophilic non-volatile liquids whose vapour pressure is below 10 mm Hg at atmospheric pressure and normal skin temperature of 32 degrees Celsius. Preferably, the dermal penetration enhancer has a molecular weight within the range of 200 to 400 Daltons.

The dermal penetration enhancers may be selected from the group consisting of fatty acids, fatty acid esters, fatty alcohols, glycols and glycol esters, 1,3-dioxolanes and 1,3-dioxanes, macrocyclic ketones containing at least 12 carbon atoms, oxazolidinones and oxazolidinone derivatives, alkyl-2-(N,N-disubstituted amino)-alkanoate esters, (N,N-disubstituted amino)-alkanol alkanoates, sunscreen esters and mixtures thereof. More preferably the dermal penetration enhancer is selected from the list including oleic acid, oleyl alcohol, cyclopentadecanone (CPE-218™), sorbitan monooleate, glycerol monooleate, propylene glycol monolaurate, polyethylene glycol monolaurate, 2-n-nonyl 1,3-dioxolane (SEPA™), dodecyl 2-(N,N-dimethylamino)-propionate (DDAIP) or its salt derivatives, 2-ethylhexyl 2-ethylhexanoate, isopropyl myristate, dimethyl isosorbide, 4-decyloxazolidinon-2-one (SR-38™, TCPI, Inc.), 3-methyl-4-decyloxazolidinon-2-one, octyl dimethyl-para-aminobenzoate, octyl para-methoxycinnamate, octyl salicylate and mixtures thereof.

Preferably the class of dermal penetration enhancers are safe skin-tolerant ester sunscreens.

Most preferably the ester is octyl dimethyl-para-aminobenzoate, octyl para-methoxycinnamate or octyl salicylate.

In a preferred embodiment of the invention the composition further comprises at least one estrogen.

Preferably the oestrogen is selected from the group consisting of oestradiol, oestriol, oestrone, ethinyloestradiol, mestranol, stilboestrol, dienoestrol, epioestriol, estropipate, zeranol and mixtures thereof. The most preferred estrogen is estradiol.

Other suitable estrogens are those capable of providing a similar biological response as that of estradiol when estradiol is delivered at a systemic dose in the typical range of 1 to 25 μg/day, and more preferably 5 to 20 μg/day.

The drug delivery system of the invention preferably comprises:

(i) an effective amount of at least one zinc chelating agent or prodrug thereof;

(ii) at least one non-volatile dermal penetration enhancer; and

(iii) at least one volatile liquid.

The dermal penetration enhancer is adapted to transport the zinc chelating agent across a dermal surface or mucosal membrane of an animal, including a human, when the volatile liquid evaporates, to form a reservoir or depot of a mixture comprising the penetration enhancer and the physiologically active agent or prodrug within said surface or membrane; and

The dermal penetration enhancer is of low toxicity to, and is tolerated by, the dermal surface or mucosal membrane of the animal.

It is preferred that, after application of the non-occlusive, percutaneous or transdermal drug delivery system, the folatile component of the delivery system evaporates and the area of skin to which the drug delivery system was applied becomes touch-dry. More preferably said area of skin becomes touch-dry within 3 minutes, more preferably within 1 minute.

Preferred volatile liquids of the present invention include safe skin-tolerant solvents such as ethanol and isopropanol. An aerosol propellant, such as dimethyl ether, may constitute a volatile liquid for the purpose of the present invention.

Surprisingly the group of dermal penetration compounds identified enhance the absorption of active agents and prodrugs thereof through the skin and mucous membranes while avoiding the significant pharmacological disadvantages and toxicities of prior art enhancers. Additionally, the group of compounds of the invention surprisingly exhibit appreciable penetration into and substantivity for the outer layers of the skin, namely the stratum corneum which has previously presented a formidable barrier to percutaneous drug absorption.

The drug delivery system of the present invention may be applied to the skin by means of an aerosol, spray, pump-pack, brush, swab, or other applicator. Preferably, the applicator provides either a fixed or variable metered dose application such as a metered dose aerosol, a stored-energy metered dose pump or a manual metered dose pump. In one embodiment the application is performed by means of a topical metered dose device such as aerosol.

The drug delivery system may be propelled by either pump pack or more preferably by the use of propellants such as hydrocarbons, hydro fluorocarbons, nitrogen, nitrous oxide, carbon dioxide or ethers, preferably dimethyl ether. The non-occlusive, drug delivery system is preferably in a single phase system as this allows less complicated manufacture and ease of dose uniformity. It may also be necessary to apply a number of dosages on untreated skin to obtain the desired result.

The invention will now be described with reference to the following examples. It is to be understood that the examples are provided by way of illustration of the invention and that they are in no way limiting to the scope of the invention.

### EXAMPLE 1

Enhanced skin penetration of 1,10-phenanthroline using octyl salicylate in a transdermal spray composition.

| | |
|---|---|
| Control formulation | Test formulation |

| Component | Amount | | Component | Amount | |
|---|---|---|---|---|---|
| 1,10- | 5% | w/v | 1,10- | 5% | w/v |
| phenanthroline | | | phenanthroline | | |

| | | | | |
|---|---|---|---|---|
| — | — | Octyl salicylate | 5% | w/v |

| | | | |
|---|---|---|---|
| Aqueous ethanol | to 100 ml | Aqueous ethanol | to 100 ml |
| (95%) v/v) | | (95% v/v) | |

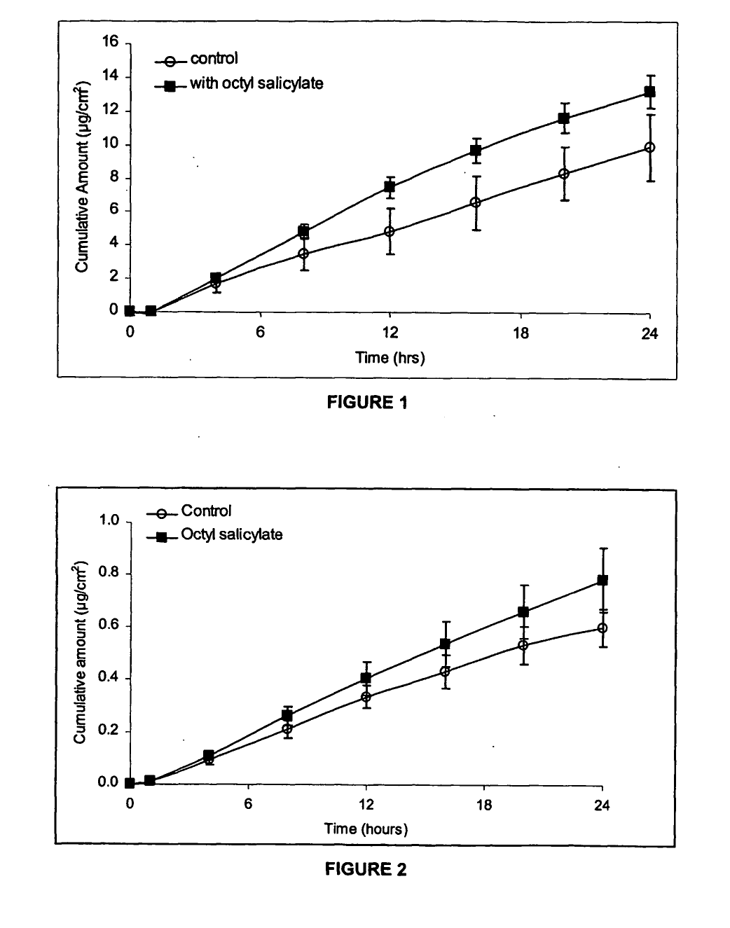

As shown in FIG. 1 the addition of the safe sunscreen ester dermal penetration enhancer, octyl salicylate (octisalate), caused a marked 1.3-fold increase in the transderrnal delivery of 1,10-phenanthroline across the skin (p<0.01).

The diffusion experiments were performed using excised human epidermis as the model membrane. These experiments were performed over 24 h with stainless steel, flow-through diffusion cells based on those previously described, (Cooper, E. R. J. Pharm. Sci. 1984, 73, 1153-1156.) except that the cell was modified to increase the diffusional area to 1.0 cm². The formulations were applied using a finite dose technique (Franz, T. J. Curr. Probl. Dermatol. 1978, 7, 58-68.) to mimic clinical dosing conditions at an applied dose volume of 5 μL/cm². A piece of stainless steel wire mesh was placed directly below the skin in the receptor chamber of the diffusion cell to maintain a turbulent flow of receptor solution below the skin. The diffusion cells were maintained at a flow rate of approximately 1.0 ml/cm²/h by a microcassette peristaltic pump (Watson Marlow 505S, UK). The cells were kept at 32±0.5° C. by a heater bar and the samples are collected into appropriately sized plastic vials on an automated fraction collector (Isco Retriever II, Lincoln, Nebr.) at specified intervals. The receptor solution (20% ethanol, 0.1% w/v sodium azide in dilute phosphate buffer) maintained sink conditions beneath the skin.

Samples were analysed for 1,10-phenanthroline directly by RP-HPLC using the following conditions; Column—Waters Symmetry C₁₈ column (3.9×150 mm) with a 5 μm support size; Mobile phase—20% Acetonitrile buffered with 0.01 M KH₂PO₄, pH=2.80; Flow rate—0.9 mL/min; Absorbance—235 nm; and Injection volume—20 μL.

### EXAMPLE 2

Enhanced skin penetration of estradiol using other safe sunscreen ester dermal penetration enhancers in a transdermal spray composition.

| Control formulation | | | Test formulation | | |
|---|---|---|---|---|---|
| Component | Amount | | Component | Amount | |
| Estradiol | 1.43% | w/v | Estradiol | 1.43% | w/v |

| | | | | |
|---|---|---|---|---|
| — | — | Octyl salicylate | 5% | w/v |

| | | | |
|---|---|---|---|
| Aqueous ethanol | to 100 ml | Aqueous ethanol | to 100 ml |
| (95%) v/v) | | (95% v/v) | |

The diffusion experiments were performed according to example 1.

As shown in FIG. 2 the addition of the safe sunscreen ester dermal 5 penetration enhancer, octyl salicylate (octisalate), surprisingly caused a marked 1.3-fold increase in the transdermal delivery of estradiol across the skin (p<0.05).

### EXAMPLE 3

Combined transdermal spray composition

| Component | Amount (% w/v) |
|---|---|
| 1,10-phenanthroline | 5.0 |
| Estradiol | 0.5 |
| Octyl salicylate | 5.0 |
| Ethanol 95% | to volume |

### EXAMPLE 4

Combined transdermal spray composition

| Component | Amount (% w/v) |
|---|---|
| 8-hydroxy quinoline | 5.0 |
| Estradiol | 0.5 |
| Isopropyl myristate | 10.0 |
| Alcohol USP (95%) | to volume |

### EXAMPLE 5

Transdermal gel composition

| Composition 1 | | Composition 2 | |
|---|---|---|---|
| | Amount | | Amount |
| Component | (% w/w) | Component | (% w/w) |
| 1,10-phenanthroline | 2 | 1,10-phenanthroline | 2 |
| Octyl salicylate | 2 | Isopropyl myristate | 2 |
| Carbomer | 0.9 | Carbomer | 0.9 |
| 0.1N NaOH | 4.72 | 0.1N NaOH | 4.72 |
| Aqueous ethanol | to 100 g | Aqueous ethanol | to 100 g |
| (95% v/v) | | (95% v/v) | |

### EXAMPLE 6

Combined transdermal gel composition

| Component | Amount (% w/w) |
|---|---|
| 1,10-phenanthroline | 0.2 |
| Estradiol | 0.2 |
| Octyl salicylate | 2.0 |
| Ethoxy cellulose | 1.0 |
| Aqueous ethanol (95% v/v) | 70% |
| Water | to volume |

### EXAMPLE 7

Enhanced matrix-type transderrnal patch compositions

| Composition 1 | | Composition 2 | |
|---|---|---|---|
| | Amount | | Amount |
| Component | (% w/w) | Component | (% w/w) |
| Ibuprofen | 2 | 1,10-phenanthroline | 2 |
| Octyl salicylate | 2 | Padimate O | 1.5 |
| Antioxidant | 0.5 | Antioxidant | 0.5 |
| Solubilizing agent | 12.75 | Solubilizing agent | 12.75 |
| Acrylic resin | 2.5 | Acrylic resin | 3 |
| Ethyl cellulose | 0.25 | Ethyl cellulose | 0.25 |
| Surfactant | 20 | Surfactant | 20 |
| Pressure sensitive | 60 | Pressure sensitive | 60 |
| adhesive | | adhesive | |

### EXAMPLE 8

Enhanced transmucosal (buccal) spray compositions

| Component | Amount (% w/v) |
|---|---|
| 1,10-phenanthroline | 5.0 |
| Estradiol | 0.5 |
| Enhancers | to 10.0 |
| Flavouring Agents | to 0.5 |
| Ethanol 70% | to volume |

### EXAMPLE 9

Combined transdermal cream composition

| Ingredient | Amount (% w/w) |
|---|---|
| Estradiol | 0.2 |
| Phenanthroline | 0.2 |
| Octyl salicylate | 2.0 |
| Propylene glycol | 6.0 |
| Cetearyl alcohol | 5.0 |
| Pyrollidine carboxylic acid (PCA) | 5.0 |
| Capric/caprylic triglycerides | 3.0 |
| Glyceryl stearate (non-self emulsifying) | 3.0 |
| Dimethicone (100 cs) | 2.0 |
| PEG 40 stearate | 2.0 |
| Phenonip | 1.0 |
| Shea butter | 1.0 |
| Crill 3 | 0.5 |
| Tocopherol | 0.5 |
| Xanthan gum | 0.35 |
| Fragrance | 1.5 |
| Water | to volume |

1. A method of treatment or prophylaxis of amyloidosis disorders in a patient the method comprising topically applying to an area of skin of the patient a composition comprising:
one or more zinc chelators; and
one or more dermal penetration enhancers.

2. A method according to claim 1 wherein the composition further comprises a volatile pharmaceutically acceptable solvent.

3. A method according to claim 1 wherein the composition further comprises one or more estrogens.

4. A method according to claim 1 wherein the composition further comprises estradiol.

5. A method according to claim 1 wherein the composition is in a form selected from the group consisting of gels, lotions, sprays compositions and patches.

6. A method according to claim 1 wherein the composition is in the form of a spray composition.

7. A method according to claim 6 wherein the composition is applied by spraying the composition onto the skin of the patient.

8. A method according to claim 1 wherein the composition comprises one or more other components selected from the group consisting of active agents, co-solvents, surfactants, emulsifiers, antioxidants, preservatives, stabilisers, diluents and mixtures of two or more thereof.

9. A composition according to claim 1 wherein at least one of a co-solvent and surfactant are present to maintain the zinc chelating agent in solution or suspension at the concentration used.

10. A method according to claim 1 wherein the transdermal administration provides a sustained low dose of zinc chelator for reducing or preventing Aβ deposits.

11. A method according to claim 1 wherein the composition comprises in the range of from about 0.1% to about 10% of a zinc chelator, from about 0.1% to about 10% of a dermal penetration enhancer, and from about 45% to about 99.8% of a volatile solvent.

12. A method according to claim 11 wherein the volatile liquid is selected from the group consisting of ethanol, isopropanol and mixture thereof.

13. A method according to claim 1 wherein the composition comprises 1 to 5% of a zinc chelator, from about 2 to 8% of the dermal penetration enhancer, from about 45 to 90% ethanol, isopropanol or mixture thereof, and from 5 to 45% water.

14. A method according to claim 11 wherein the composition further comprises a thickening agent.

15. A method according to claim 1 wherein the composition comprises from 0.1 to 2% oestrogen.

16. A method according to claim 1 wherein the zinc chelator has a molecular weight less than 500 Daltons, a melting point less than 200 degrees Celsius, less than or equal to 3 hydrogen bond donors, an octanol-water partition coefficient between 1 and 4 and a water solubility greater than 10 microgram per millilitre.

17. A method according to claim 1 wherein the zinc chelator is selected from phenanthrolines and their derivatives.

18. A method according to claim 1 wherein the zinc chelator has a chemical binding site or sites(s) for a zinc ion as determined by a negative binding energy of greater than 20 kcal/mole for the association of the zinc ion and the compound of interest when using a recognised 3-dimensional molecular modelling software such as “ChemDraw” 3D, version 5.0 running a MM2 force-field for the steric energy calculation.

19. A method according to claim 1 wherein the zinc chelator comprises at least one selected from the group consisting of 3-mercapto-D valine, bis(diethylthiocarbamoyl) disulfide, N,N,N′,N′-tetrakis (2-pyridylmethyl)-ethylenediamine,N-(6-methoxy-8-quinolyl)-p-toluenesulfonamide, 8-hydroxy quinoline, 8-hyroxy quinoline-5-sulphonic acid, diethyl dithiocarbamate, phenanthroline and it's derivatives, dipicolinate, diphenylthiocarbazone, dithizone, cimetidine, dipicolinic acid, clioquinol or pharmaceutically acceptable salts or derivatives of any one of the aforementioned.

20. A method according to claim 1 wherein the zinc chelator is selected from the group consisting of diclofenac, ibuprofen, naproxen, piroxicam, indomethacin, ketoprofen, nabumetone, apazone, sulindac, meloxicam, tiaprofenic acid, flurbiprofen, tolfenamic acid, phenylbutazone, benzydamide, aspirin, salicylic acid and pharmaceutically acceptable salts and derivatives thereof.

21. A method according to claim 1 wherein the release rate profile of the chelating agent into the systemic circulation is approaching zero order in nature whereby the potential side effects associated with elevated maximum concentration (Cₘₐₓ) to average concentration (C_{avg}) ratios with oral administration are reduced.

22. A method according to claim 1 wherein the method provides a therapeutically effective blood serum level over 12 hours.

23. A method according to claim 1 wherein the dermal penetration enhancer is selected from the group consisting of fatty acids, fatty acid esters, fatty alcohols, glycols and glycol esters, 1,3-dioxolanes and 1,3-dioxanes, macrocyclic ketones containing at least 12 carbon atoms, oxazolidinones and oxazolidinone derivatives, alkyl-2-(N,N-disubstituted amino)-alkanoate esters, (N,N-disubstituted amino)-alkanol alkanoates, sunscreen esters and mixtures thereof.

24. A method according to claim 1 wherein the dermal penetration enhancer is selected from the group consisting of oleic acid, oleyl alcohol, cyclopentadecanone (CPE-218™), sorbitan monooleate, glycerol monooleate, propylene glycol monolaurate, polyethylene glycol monolaurate, 2-n-nonyl 1,3-dioxolane (SEPA™), dodecyl 2-(N,N-dimethylamino)-propionate (DDAIP) or its salt derivatives, 2-ethylhexyl 2-ethylhexanoate, isopropyl myristate, dimethyl isosorbide, 4-decyloxazolidinon-2-one (SR-38™, TCPI, Inc.), 3-methyl-4- decyloxazolidinon-2-one, octyl dimethyl-para-aminobenzoate, octyl para-methoxycinnamate, octyl salicylate and mixtures thereof.

25. A method according to claim 1 wherein the penetration enhancer is selected from safe skin-tolerant ester sunscreens.

26. A method according to claim 24 wherein the safe skin-tolerant ester sunscreens are selected from the group consisting of octyl dimethyl-para-aminobenzoate, octyl para-methoxycinnamate or octyl salicylate.

27. A method according to claim 3 wherein the estrogen is selected from the group consisting of oestradiol, oestriol, oestrone, ethinyloestradiol, mestranol, stilboestrol, dienoestrol, epioestriol, estropipate, zeranol and mixtures thereof.

28. A transdermal composition for the treatment or prophylaxis of amyloidosis disorders in a patient the transdermal composition comprising:
one or more zinc chelators; and
one or more dermal penetration enhancers.

29. A transdermal composition according to claim 28 wherein the composition further comprises a volatile pharmaceutically acceptable solvent.

30. A transdermal composition according to claim 28 further comprising one or more estrogens.

31. A transdermal composition according to claim 28 further comprising estradiol.

32. A transdermal composition according to claim 28 in a form selected from the group consisting of gels, lotions, spray compositions and patches.

33. A transdermal composition according to claim 28 in the form of a spray composition.

34. A transdermal composition according to claim 28 comprising one or more other components selected from the group consisting of active agents, co-solvents, surfactants, emulsifiers, antioxidants, preservatives, stabilisers, diluents and mixtures of two or more thereof.

35. A transdermal composition according to claim 28 comprising at least one of a co-solvent and surfactant present in an amount to maintain the zinc chelating agent in solution or suspension at the concentration used.

36. A transdermal composition according to claim 28 wherein the transdermal composition provides a sustained low dose of zinc chelator for reducing or preventing Aβ deposits.

37. A transdermal composition according to claim 28 comprising in the range of from about 0.1% to about 10% of a zinc chelator, from about 0.1% to about 10% of a dermal penetration enhancer, and from about 45% to about 99.8% of a volatile solvent.

38. A transdermal composition according to claim 37 wherein the volatile solvent is selected from the group consisting of ethanol, isopropanol and mixture thereof.

39. A transdermal composition according to claim 28 comprising 1 to 5% of a zinc chelator, from about 2 to 8% of the dermal penetration enhancer, from about 45 to 90% ethanol, isopropanol or mixture thereof, and from 5 to 45% water.

40. A transdermal composition according to claim 39 further comprising a thickening agent.

41. A transdermal composition according to claim 28 further comprising from 0.1 to 2% oestrogen.

42. A transdermal composition according to claim 28 wherein the zinc chelator has a molecular weight less than 500 Daltons, a melting point less than 200 degrees Celsius, less than or equal to 3 hydrogen bond donors, an octanol-water partition coefficient between 1 and 4 and a water solubility greater than 10 microgram per millilitre.

43. A transdermal composition according to claim 28 wherein the zinc chelator is selected from phenanthrolines and their derivatives.

44. A transdermal composition according to claim 28 wherein the zinc chelator has a chemical binding site or sites(s) for a zinc ion as determined by a negative binding energy of greater than 20 kcal/mole for the association of the zinc ion and the compound of interest when using a recognised 3-dimensional molecular modelling software such as “ChemDraw” 3D, version 5.0 running a MM2 force-field for the steric energy calculation.

45. A transdermal composition according to claim 28 wherein the zinc chelating agent is selected from the group consisting of 3-mercapto-D valine, bis(diethylthiocarbamoyl) disulfide, N,N,N′,N′-tetrakis (2-pyridylmethyl)-ethylenediamine,N-(6-methoxy-8-quinolyl)-p-toluenesulfonamide, 8-hydroxy quinoline, 8-hyroxy quinoline-5-sulphonic acid, diethyl dithiocarbamate, phenanthroline and it's derivatives, dipicolinate, diphenylthiocarbazone, dithizone, cimetidine, dipicolinic acid, clioquinol and pharmaceutically acceptable salts and derivatives and mixtures of any one of the aforementioned.

46. A transdermal composition according to claim 28 wherein the zinc chelator is selected from the group consisting of diclofenac, ibuprofen, naproxen, piroxicam, indomethacin, ketoprofen, nabumetone, apazone, sulindac, meloxicam, tiaprofenic acid, flurbiprofen, tolfenamic acid, phenylbutazone, benzydamide, aspirin, salicylic acid and pharmaceutically acceptable salts and derivatives thereof.

47. A transdermal composition according to claim 28 wherein the release rate profile of the chelating agent into the systemic circulation is approaching zero order in nature whereby the potential side effects associated with elevated maximum concentration (Cₘₐₓ) to average concentration (C_{avg}) ratios with oral administration are reduced.

48. A transdermal composition according to claim 28 wherein the dermal penetration enhancer is selected from the group consisting of fatty acids, fatty acid esters, fatty alcohols, glycols and glycol esters, 1,3-dioxolanes and 1,3-dioxanes, macrocyclic ketones containing at least 12 carbon atoms, oxazolidinones and oxazolidinone derivatives, alkyl-2-(N,N-disubstituted amino)-alkanoate esters, (N,N-disubstituted amino)-alkanol alkanoates, sunscreen esters and mixtures thereof.

49. A transdermal composition according to claim 28 wherein the dermal penetration enhancer is selected from the group consisting of oleic acid, oleyl alcohol, cyclopentadecanone (CPE-218™), sorbitan monooleate, glycerol monooleate, propylene glycol monolaurate, polyethylene glycol monolaurate, 2-n-nonyl 1,3-dioxolane (SEPA™), dodecyl 2-(N,N-dimethylamino)-propionate (DDAIP) or its salt derivatives, 2-ethylhexyl 2-ethylhexanoate, isopropyl myristate, dimethyl isosorbide, 4-decyloxazolidinon-2-one (SR-38™, TCPI, Inc.), 3-methyl-4- decyloxazolidinon-2-one, octyl dimethyl-para-aminobenzoate, octyl para-methoxycinnamate, octyl salicylate and mixtures thereof.

50. A transdermal composition according to claim 28 wherein the penetration enhancer is selected from safe skin-tolerant ester sunscreens.

51. A transdermal composition according to claim 28 wherein the safe skin-tolerant ester sunscreens are selected from the group consisting of octyl dimethyl-para-aminobenzoate, octyl para-methoxycinnamate or octyl salicylate.

52. A transdermal composition according to claim 31 wherein the estrogen is selected from the group consisting of oestradiol, oestriol, oestrone, ethinyloestradiol, mestranol, stilboestrol, dienoestrol, epioestriol, estropipate, zeranol and mixtures thereof.
